(19) United States
(12) Patent Application Publication
(10) Pub. No.: US 2002/0025929 A1
(43) Pub. Date: Feb. 28, 2002

(54) METHOD OF BUILDING AND MAINTAINING BONE

(76) Inventor: MASAHIKO SATO, CARMEL, IN (US)

Correspondence Address: WILLIAM R BOUDREAUX, ELI LILLY AND COMPANY, PATENT DIVISION DC: 1104, LILLY COMPANY CENTER, INDIANAPOLIS, IN, 46285

(21) Appl. No.: 09/169,786

(22) Filed: Oct. 9, 1998

Related U.S. Application Data
(60) Provisional application No. 60/061,800, filed on Oct. 14, 1997.

(51) Int. Cl. A61K 38/00; C07K 5/00; C07K 7/00; C07K 17/00; C07K 16/00; C07K 2/00; C07K 4/00; C07K 14/00; A01N 43/12; A61K 31/38; A01N 43/40; A01N 43/42

(52) U.S. Cl. 514/12; 530/324; 530/300; 514/299; 514/337; 514/443

(57) ABSTRACT

A method of treating bone loss comprises co-administering therapeutically effective amounts of parathyroid hormone and a compound of the formula

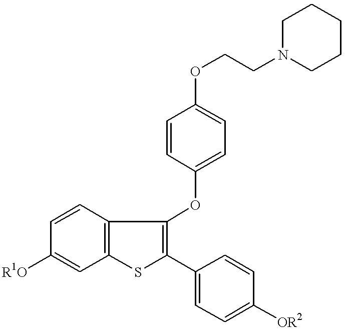

or a pharmaceutically acceptable salt thereof wherein R¹ and R² are independently selected from the group consisting of hydrogen and alkyl of one to six carbon atoms.

The co-administration may take the mode of simultaneous, concurrent, or sequential administration of the two compounds.

### TECHNICAL FIELD

The present invention relates to medical methods of treatment. More particularly, the invention concerns the use of certain 3-(substituted phenoxy)benzo[b]thiophene compounds in combination with parathyroid hormone (PTH) for the treatment of patients deficient in mineralized tissue.

### BACKGROUND OF THE INVENTION

Osteoporosis is a condition observed for postmenopausal women, generally aged 65 years or more, and for men aged 80 years or older. In women, osteoporosis is observed primarily with the decline of ovarian function at about 45 years of age. In men and women, osteoporosis can also result from treatment with immunosuppressants, steroids (glucocorticoids, corticosteroids), diabetes, hypogonadism, hyperparathyroidism, arthritis (rheumatoid and osteoarthritis), and behavioral choices (smoking, drinking, diet).

The condition is characterized by low bone mass and is due either to excessive bone resorption or a decrease in bone formation activity. Either mechanism results in a net decline in bone mass and bone density with an attendant increased risk of bone fractures in the patient.

Existing methods of treatment of osteoporosis are aimed primarily at preventing the loss of bone mass by increasing calcium intake in the diet and/or by inhibiting the activity of the bone resorbing cells (osteoclasts). Specifically, present treatment modalities, including estrogen replacement therapy, calcitonin, or bisphosphonates, all inhibit bone resorption by inhibiting the activity or differentiation of osteoclasts.

Parathyroid hormone (PTH, Sequence ID No. 1) is a linear polypeptide (Mᵣ 9500) containing 84 amino acid residues which is excreted from the parathyroid gland in response to low Ca⁺⁺ levels in serum. Although the excreted peptide is 84 amino acid residues in length, the anabolic activity of the hormone can be performed by the N-terminal 31 or 34 residue fragments. Physiologically, PTH maintains normal levels of serum calcium concentration by interacting with bone, kidney, and the intestine (indirectly through the vitamin D axis).

Chronically increased levels of PTH, as observed in hyperparathyroidism, results in the loss of bone and can often result in renal calculi and the deposition of calcium phosphate in soft tissue. However, clinical data have shown that intermittent, subcutaneous, daily injections of hPTH (1-34) increases bone mass in the absence of hypercalcemia. (Cf. D. Dempster, et al., Endocrine Rev., 14: 690-709 (1993); R. Lindsay, et al., Lancet, 350: 550 ff., 1997; and F. Cosman and R. Lindsay, Calcif. Tissue Int., 62: 475-480 (1998)).

U.S. Pat. No. 5,118,667 to Adams, et al. discloses the use of PTH as a bone growth factor and as an inhibitor of bone resorption.

Smaller fragments of the full 84 residue hPTH have also been shown to stimulate bone formation. These include the 1-31 N-terminal fragment, (hPTH(1-31)NH₂, ostabolin), reported by J. F. Whitfield, et al., J. Bone & Min. Res., 12(8): 1246-1252 (1997); and the 1-34 N-terminal fragment reported by G. W. Tregear, Hoppe-Seyler's Z. Physiol. Chem., 355: 415-421; and R. Lindsay, et al., op. cit.

U.S. Pat. No. 5,510,370 to Hock discloses the use of parathyroid hormone together with the compound generically known as raloxifene for increasing bone mass.

### BRIEF DESCRIPTION OF THE DRAWING

IN THE DRAWING, FIG. 1 is a graph showing the effects upon the proximal tibia in the ovariectomized rate model of treatment in accordance with the method of the present invention in comparison with control experiments.

FIG. 2 is a series of graphs showing the effects upon the distal femur metaphysis in osteopenic rats of treatment in accordance with the method of the present invention in comparison with control experiments.

FIG. 3 is a series of graphs showing the effects upon the L-3 vertebra of osteopenic rats of treatment in accordance with the method of the present invention in comparison with control experiments.

### SUMMARY OF THE INVENTION

In its principal embodiment, the present invention provides a method of building bone mass in a patient in need of such treatment comprising administering a therapeutically effective amount of a compound of formula 1:

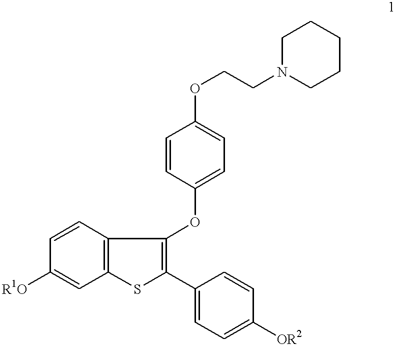

where R¹ and R² are independently hydrogen or alkyl of one to four carbon atoms, or a pharmaceutically acceptable salt thereof, in combination with PTH or a pharmaceutically acceptable salt thereof.

### DETAILED DESCRIPTION

In accordance with the present invention, it has been found that administration of both PTH and a compound of formula 1 more effectively treats bone loss than does administration of either compound alone.

As used throughout this specification and the appended claims, “co-administration” of a compound of Formula 1 with PTH or “administration” of a compound of formula 1 “with” PTH means the simultaneous, concurrent, or sequential administration of the two compounds for treating conditions characterized by insufficient bone.

By “simultaneous administration” is meant the administration of therapeutically effective doses of PTH and a compound of Formula 1 in a single unit dosage form.

“Concurrent administration” means the administration of therapeutically effective amounts of PTH and a compound of Formula 1 in separate unit dosage forms within a short period of one another, essentially administering the two drugs “at the same time” but in separate dosage forms. This mode of administration permits the administration of PTH in one dosage form, such as an iontophoretic transdermal patch, an oral, pulmonary or nasal spray, sub-cutaneous, parenteral, buccal, or sub-lingual or suppository dosage form, and the administration of a compound of Formula 1 in another such as any of the foregoing dosage forms or in an oral dosage form such as a tablet, capsule, syrup or elixir, as well as by means of a suppository.

“Sequential administration” means the administration of a therapeutically effective amount of either PTH or a compound of Formula 1 alone, after which administration of the one compound is halted and administration of the other compound is begun. Sequential administration may also take the form of simultaneous or concurrent administration of the two drugs, followed by cessation of the simultaneous or concurrent administration of the two drugs and continued administration of either compound alone.

By the term “PTH” or “parathyroid hormone” as used throughout this specification and claims is meant any polypeptide, protein, protein fragment, or modified protein fragment capable of mimicking the activity of human parathyroid hormone (1-84) in controlling calcium and phosphate metabolism to build bone in the human body. Included within this definition are the full 84 amino acid sequence of human parathyroid hormone (hPTH, Sequence ID No. 1); the 1-31 residue N-terminal fragment of human parathyroid hormone (hPTH 1-31, Sequence ID No. 2); the 1-34 residue N-terminal fragment of human parathyroid hormone (hPTH 1-34, Sequence ID No. 3); the 1-38 residue N-terminal fragment of human parathyroid hormone (hPTH 1-38, Sequence ID No. 4); PTH related peptide (PTHrP 1-34, Sequence ID No. 5); [Glu²², Leu²³, Glu²⁵, Lys²⁶, Leu²⁸, Glu²⁹, Lys³⁰, Leu³¹, homo-Ser³⁴ (lactam)]PTHrP (Sequence ID No. 6); [Ala²¹, Glu²², Leu²³, Glu²⁵ Lys²⁶, Leu²⁸, Glu²⁹, Lys³⁰, Leu³¹]PTHrP (Sequence ID No. 7); [Glu²², Leu²³, Glu²⁵, Lys²⁶, Leu²⁸, Glu²⁹, Lys³⁰, His³¹, Thr³², Ala³⁴, des-Ala³⁴]PTHrP (Sequence ID No. 8); [Leu²⁷ cyclo(Glu²²-Lys²⁶)]hPTH(1-34) (Sequence ID No. 9); Leu²⁷ cyclo(Lys²⁶-Asp³⁰)]hPTH(1-31) (Sequence ID No. 10; and [Leu⁸, Asp¹⁰, Lys¹¹, Ala¹⁶, Gln¹⁸, Thr³³, Ala³⁴]hPTH(1-34) (Sequence ID No. 11); and PTHrP 1-36 (Sequence ID no. 12).

The preferred “PTH” for use in the method of this invention is the N-terminal 1-34 fragment of human parathyroid hormone, hPTH(1-34, Sequence ID No. 3). By the term “alkyl” is meant a monovalent radical derived from methane, ethane or a branched or straight-chain saturated hydrocarbon of 3 or 4 carbon atoms by the removal of a single hydrogen atom.

The preferred compounds of formula 1 for use in the method of this invention are the compound in which R¹ is hydrogen and R² is methoxy, i.e. 6-hydroxy-2-(4-methoxyphenyl)-3-[4-(2-piperidin-1-ylethoxy)phenoxy]-benzo[b]thiophene, compound 1a:

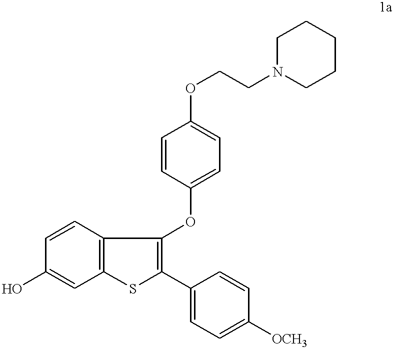

and the compound where both R¹ and R² are hydrogen, i.e. 6-hydroxy-2-(4-hydroxyphenyl)-3-[4-(2-piperidin-1-ylethoxy)phenoxy]benzo[b]thiophene, compound 1b:

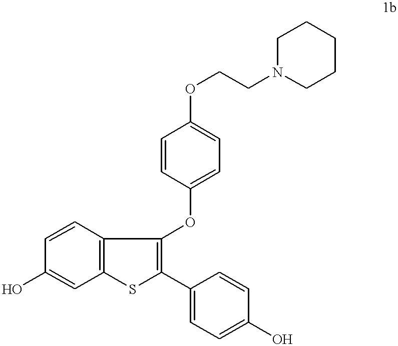

Methods for the production of compounds of Formulae 1, 1a, and 1b and their pharmaceutically acceptable salts are given in U.S. Pat. No. 5,510,357 issued Apr. 23, 1996 and U.S. Pat. No. (08/552636 filed Nov. 3, 1995). The preferred salt is the hydrochloride.

The full hPTH 1-84, and the various N-terminus fragments, (either unmodified or modified by the substitution of one or more aminoacyl residues in the fragment) can be prepared synthetically or recombinantly by techniques well known to those skilled in the art. Synthetic examples include the so-called “solid phase” peptide synthesis and usual methods of solution phase chemistry. A summary of available solid phase peptide synthetic techniques may be found in J. M. Stewart and J. D. Young, Solid Phase Peptide Synthesis, W. H. Freeman Co., San Francisco, 1963 and J. Meinhofer, Hormonal Proteins and Peptides, Vol. 2, Academic Press, New York, 1973. Classical solution synthesis techniques are described by G. Schroeder and K. Lupke, The Peptides, Vol.1, Academic Press, New York, 1965.

In general, these synthetic methods comprise the sequential addition of one or more amino acids or suitably protected amino acids to a growing peptide chain bound to a synthetic resin. The starting amino acids are commercially available.

Normally, either the amino or carboxyl function of the first amino acid is protected by a suitable protecting group. The protected or derivatized amino acid can then be either attached to an inert solid support (resin) or utilized in solution phase synthesis by adding the next amino acid in the sequence having the complementary (amino or carboxyl) group suitably protected, under conditions conducive to formation of the amide (peptide) link. The protecting group is then removed from this newly added amino acid residue and the next (suitably protected) amino acid is added, and so forth.

After all of the desired amino acids have been linked in the proper sequence, any remaining protecting groups are removed, sequentially or concurrently, and the peptide chain, if synthesized by the solid phase method, is cleaved from the solid support to afford the final polypeptide. By simple modification of this general procedure, it is possible to add more than one amino acid to the growing chain, for example, by coupling (under conditions which do not racemize chiral centers) a protected tripeptide with a properly protected dipeptide to form, after deprotection, a pentapeptide.

A particularly preferred method of preparing the peptides involves solid phase peptide synthesis. In this method, the α-amino function of the amino acid is protected by an acid or base sensitive group. Such protecting groups should have the properties of being stable to the conditions of peptide linkage formation, while being readily removable without destruction of the growing peptide chain and without causing racemization of any chiral centers contained therein.

Suitable protecting groups are tert-butoxycarbonyl (BOC), benzyloxycarbonyl (Cbz), biphenylisopropyloxycarbonyl, tert-amyloxycarbonyl, isobornyloxycarbonyl, α,α-dimethyl-3,5-dimethoxybenzyloxycarbonyl, ortho-nitrophenylsulfenyl, 2-cyano-tert-butoxycarbonyl, 9-fluorenylmethyloxycarbonyl, and the like. The tert-butoxycarbonyl (BOC) protecting group is preferred.

Particularly preferred side-chain protecting groups are, for lysine and arginine: nitro, para-toluenesulfonyl, 4-methoxybenzenesulfonyl, Cbz, BOC, and adamantyl-oxycarbonyl; for tyrosine: benzyl, ortho-bromo-benzyloxycarbonyl, 2,6idichlorobenzyl, isopropyl, cyclohexyl, cyclopentyl, and acetyl; for serine: benzyl and tetrahydropyranyl; for histidine: CBz, para-toluenesulfonyl and 2,4-dinitrophenyl; for tryptophan: formyl.

In the solid phase method, the suitably protected C-terminal amino acid is attached to a solid support. Suitable solid supports useful for this method are those materials which are inert to the reagents and reaction conditions of the stepwise protection/deprotection reactions, as well as being insoluble in the solvent medium used. Suitable solid supports are chloromethyl-polystyrene-divinylbenzene copolymer and benzhydrylamino-polystyrene-divinylbenzene copolymer described by P. Rivaille, et al., Helv. Chim. Acta, 54: 2772 (1971). Chloromethyl-polystyrene-1%-divinylbenzene copolymer is particularly preferred.

The coupling of the first, protected, amino acid residue to the chloromethyl copolymer is made by means of the reaction of its cesium, tetramethylammonium, 1,5-diazabicyclo[5.4.0]-undec-5-ene, or similar salt with the polymer resin. The reaction is typically carried out in a solvent such as ethanol, acetonitrile, N,N-dimethyl-formamide, or the like at an elevated temperature, typically between about 40° C. and 60° C. for a period of from about 12 to about 48 hours. Preferred reaction conditions involve the coupling of the protected amino acid to the resin in dimethylformamide at about 50° C. for about 24 hours.

The first, protected, amino acid is attached to the benzhydrylamin copolymer resin in the presence of a coupling reagent such as N,N-dicyclohexylvarbodiimide (DCC) or N,N′-diisopropylcarbodiimide (DIC) with or without 1-hydroxy-benzotriazole (HOBt), benzotriazol-1-yloxy-tris-(dimethyl-aino)phosphonium hexafluorophosphae (BOP) or bis-(2-oxo-3-oxazolidinyl)phosphine chloride (BOPCl). The reaction is carried out at a temperature ranging between about 10° C. and 50° C., most preferably at about 25° C. in a solvent such as dichloromethane or DMF for a period ranging between 1 and 24 hours.

The coupling of successive protected amino acids can be carried out manually or in a commercially available automated peptide synthesizer. The removal of the α-N-protecting groups may be performed, for example, in the presence of a solution of trifluoroacetic acid in methylene chloride, hydrogen chloride in dioxane, hydrogen chloride in acetic acid, or other strong acid solution, preferably 50% trifluoroacetic acid in dichloromethane at ambient temperature.

Each protected amino acid is preferably introduced in 0.4 M concentration in about 3.5 molar excess, and the coupling can be carried out in dichloromethane, dichloromethane/DMF mixtures, DMF or the like, preferably in dichloromethane at ambient temperature. The coupling reagent is normally DCC in dichloromethane, but may be DIC or other carbodiimide, either alone or in combination with HOBt, N-hydroxysuccinimide or other N-hydroxyimide or oxime. Alternatively, protected amino acids which have been activated by conversion of the carboxyl group to an active ester by reaction with para-nitrophenol, pentafluorophenol, and the like.

In addition, the full hPTH 1-84, and the various N-terminus fragments may be made recombinantly according to U.S. Pat. No. 5,605,815, issued Feb. 25, 1997; U.S. Pat. No. 5,420,242, issued May 30, 1995; PCT Intnl. Publ. No. WO 96/36721, published Nov. 21, 1996; European Pat. Appln. Publ. No. 0 499 990 A2, published Aug. 26, 1992; European Pat. Appln. Publ. No. 0 483 509 A1, published May 6, 1992; J. Paulsen et al., J. Biotech. 39, 126-131 (1995); Y. Susuki et al., Appl. Environ. Microbiol. 64(2), 526-529 (1998); and H. Gramm et al., Bio/Technology 12, 1017-1023 (1994).

### Pharmaceutical Formulations

The present invention also provides pharmaceutical compositions which comprise compounds of the present invention formulated together with one or more non-toxic pharmaceutically acceptable carriers and/or excipients. The formulations may be specially formulated for transdermal administration by means of an iontophoretic patch, for oral administration, in solid or liquid form, for parenteral injection, or for rectal or vaginal administration by means of a suppository.

The pharmaceutical compositions of this invention can be administered to humans and other mammals orally, rectally, intravaginally, parenterally, topically (by means of powders, ointments, creams, drops or patches), buccally or sublingually, or as an oral, pulmonary, or nasal spray. The term “parenteral administration” refers herein to modes of administration which include intravenous, intramuscular, intraperitoneal, intrasternal, subcutaneous, or intraarticular injection or infusion.

Pharmaceutical compositions of this invention for parenteral administration comprise sterile aqueous or non-aqueous solutions, dispersions, suspensions, or emulsions, as well as sterile powders which are reconstituted immediately prior to use into sterile solutions or suspensions. Examples of suitable sterile aqueous and non-aqueous carriers, diluents, solvents or vehicles include water, physiological saline solution, ethanol, polyols (such as glycerol, propylene glycol, poly(ethylene glycol), and the like), and suitable mixtures thereof, vegetable oils (such as olive oil), and injectable organic esters such as ethyl oleate. Proper fluidity is maintained, for example, by the use of coating materials such as lecithin, by the maintenance of proper particle size in the case of dispersions and suspensions, and by the use of surfactants.

Parenteral compositions may also contain adjuvants such as preservatives, wetting agents, emulsifying agents, and dispersing agents. Prevention of the action of micro-organisms is ensured by the inclusion of antibacterial and antifungal agents, for example, paraben, chlorobutanol, phenol sorbic acid, and the like. It may also be desirable to include isotonic agents such as sugars, sodium chloride, and the like. Prolonged absorption of injectable formulations may be brought about by the inclusion of agents which delay absorption such as aluminum monostearate and gelatin.

In some cases, in order to prolong the effect of the drug, it is desirable to slow the absorption of the drug following subcutaneous or intramuscular injection. This may be accomplished by the use of a liquid suspension or crystalline or amorphous material of low water solubility or by dissolving or suspending the drug in an oil vehicle. In the case of the subcutaneous or intramuscular injection of a suspension containing a form of the drug with low water solubility, the rate of absorption of the drug depends upon its rate of dissolution.

Injectable “depot” formulations of the compounds of this invention are made by forming microencapsulated matrices of the drug in biodegradable polymers such as poly(lactic acid), poly(glycolic acid), copolymers of lactic and glycolic acid, poly (orthoesters), and poly (anhydrides) these materials which are described in the art. Depending upon the ratio of drug to polymer and the characteristics of the particular polymer employed, the rate of drug release can be controlled.

Injectable formulations are sterilized, for example, by filtration through bacterial-retaining filters, or by presterilization of the components of the mixture prior to their admixture, either at the time of manufacture or just prior to administration (as in the example of a dual chamber syringe package).

Solid dosage forms for oral administration include capsules, tablets, pills, powders, and granules. In such solid dosage forms, the active component is mixed with at least one inert, pharmaceutically acceptable carrier such as sodium citrate, or dicalcium phosphate, and/or (a) fillers or extenders such as starches, lactose, glucose, mannitol, and silicic acid, (b) binding agents such as carboxymethyl-cellulose, alginates, gelatin, poly(vinylpyrrolidine), sucrose and acacia, (c) humectants such as glycerol, (d) disintegrating agents such as agar-agar, calcium carbonate, potato or tapioca starch, alginic acid, silicates and sodium carbonate, (e) solution retarding agents such as paraffin, (f) absorption accelerating agents such as quaternary ammonium compounds, (g) wetting agents such as cetyl alcohol and glycerin monostearate, (h) absorbents such as kaolin and bentonite clay, and (i) lubricants such as talc, calcium stearate, magnesium stearate, solid poly(ethylene glycols), sodium lauryl sulfate, and mixtures thereof. In the case of capsules, tablets and pills, the dosage form may also contain buffering agents.

Solid compositions of a similar type may also comprise the fill in soft or hard gelatin capsules using excipients such as lactose as well as high molecular weight poly(ethylene glycols) and the like.

Solid dosage forms such as tablets, dragees, capsules, pills and granules can also be prepared with coatings or shells such as enteric coatings or other coatings well known in the pharmaceutical formulating art. The coatings may contain opacifying agents or agents which release the active ingredient(s) in a particular part of the digestive tract, as for example, acid soluble coatings for release of the active ingredient(s) in the stomach, or base soluble coatings for release of the active ingredient(s) in the intestinal tract.

The active ingredient(s) may also be microencapsulated in a sustained-release coating, with the microcapsules being made part of a pill of capsule formulation.

Liquid dosage forms for oral administration of the compounds of this invention include solution, emulsions, suspensions, syrups and elixirs. In addition to the active components, liquid formulations may include inert diluents commonly used in the art such as water or other pharmaceutically acceptable solvents, solubilizing agents and emulsifiers such as ethanol, isopropanol, ethyl carbonate, ethyl acetate, benzyl alcohol, benzyl benzoate, propylene glycol, 1,3-butylene glycol, dimethyl formamide, oils (in particular, cottonseed, ground nut, corn, germ, olive, castor, and sesame oils), glycerol, tetrahydrofurfuryl alcohol, poly(ethylene glycols), fatty acid esters of sorbitol, and mixtures thereof.

Besides inert diluents, the liquid oral formulations may also include adjuvants such as wetting agents, emulsifying and suspending agents, and sweetening, flavoring, and perfuming agents.

Liquid suspension, in addition to the active ingredient(s) may contain suspending agents such as ethoxylated isostearyl alcohols, polyoxyethylene sorbitol and sorbitan esters, microcrystalline cellulose, aluminum metahydroxide, bentonite clay, agar-agar, and tragacanth, and mixtures thereof.

Compositions for rectal or intravaginal administration are prepared by mixing one or more compounds of the present invention with suitable non-irritating excipients such as cocoa butter, polyethylene glycol or any suppository wax which is a solid at room temperature, but liquid at body temperature and therefore melt in the rectum or vaginal cavity to release the active component(s). The compounds are dissolved in the melted wax, formed into the desired shape, and allowed to harden into the finished suppository formulation.

Compounds of the present invention may also be administered in the form of liposomes. As is know in the art, liposomes are generally derived from phospholipids or other lipid substances. Lipososome formulations are formed by mono- or multilamellar hydrated liquid crystals which are dispersed in an aqueous medium. Any non-toxic, pharmaceutically acceptable, and metabolizable lipid capable of forming liposomes can be used. The present compositions in liposome form can contain, in addition to one or more active compounds of the present invention, stabilizers, excipients, preservatives, and the like. The preferred lipids are phospholipids and the phosphatidyl cholines (lecithins), both natural and synthetic.

Methods for forming liposomes are know in the art as described, for example, in Prescott, Ed., Methods in Cell Biology, Volume XIV, Academic Press, New York, N.Y. (1976), p. 33 et seq.

Dosage forms for topical administration of the compounds of the present invention include powders, sprays, ointments, creams, and inhalants. The active ingredient(s) is mixed under sterile conditions with a suitable pharmaceutically acceptable carrier and preservatives, buffers, or propellants as needed. Opthalmic formulations, eye ointments, and solutions are also contemplated as falling within the scope of the present invention.

Actual dosage levels of compounds of the present invention are varied so as to administer an amount of the compound which is effective to bring about the desired therapeutic affect. The dose required for a given patient will vary depending upon the severity of the condition being treated, the age, weight, and sex of the patient, as well as the state of health of the patient. However, it is within the skill of the art to “dose titrate” the patient; that is, to begin administering a dose known to be below the amount required to bring about the desired therapeutic effect and to gradually increase the dose until the desired effect is achieved.

Generally, for the treatment of estrogen-related disorders, compounds of the present invention are administered at dosage levels between about 10 μg/kg of body weight to about 10 mg/kg of body weight per day. If desired, the daily dosage may be divided into multiple doses for purposes of administration, e.g. into two to four doses per day.

### Mode of Administration

PTH is co-administered with a compound of Formula 1 in accordance with the method of the present invention in a total daily dose ranging between about 5 μg and about 200 μg of PTH. The daily dose of a compound of Formula 1, preferably compound 1a, ranges between about 1 mg to about 100 mg per day, preferably between about 3 mg and 10 mg per day.

PTH is typically administered parenterally, most conveniently by means of a sub-cutaneous dose. Alternative parenteral routes of administration of PTH include intramuscular or intraperitoneal injection. The intramuscular dose may be in the form of a “depot” formulation of the type known in the art which deposits the protein, encapsulated in biodegradeable microspheres in the muscle tissue.

A convenient mode for the simultaneous administration of PTH and a compound of Formula 1 is by the subcutaneous injection of individual solutions of the two compounds contained in a multi-cartridge medication injection device of the type described in U.S. Pat. No. 5,584,815 to Pawelka, et al.

PTH and a compound of Formula 1 are also administered concurrently, for example by parenteral administration or by buccal administration to the patient by means of a lozenge which is dissolved next to the cheek, or sub-lingually by means of a lozenge or liquid drops placed under the tongue. In each of these examples of concurrent administration, the compound of Formula 1, preferably 1a, is administered in a separated dosage form.

The preferred mode of sequential administration comprises administering a combination of PTH and compound 1a by simultaneous or concurrent means for a period sufficient to raise the bone mass and or bone density of the patient to within one standard deviation of norm. This typically requires a period of co-administration ranging between 6 and 24 months, generally about 12 months. When the bone mass or bone density is determined to be within this acceptable range of normal, co-administration of both compounds is halted, and the patient is maintained on a “maintenance” regimen of a compound of formula 1, preferably compound 1a. If the circumstance arises that a subsequent loss of bone density or bone mass is detected in the patient occurs, the co-administration of PTH and the compound of Formula 1 followed by administration of a compound of Formula 1 alone is repeated.

In any of the modes of administration described above, the progress of the course of therapy is readily followed by the attending physician by periodically assessing bone mass and density by means known in the art and adjusting the dose or dosing regimen accordingly.

General methods for the measurement of bone mass and bone density and norms for these parameters are discussed by C. C. Johnston, et al., in Chapter 26, “Bone Density Measurement and the Management of Osteoporosis,” in Primer on the Metabolic Bone Diseases and Disorders of Mineral Metabolism, 2^{nd} Edition, M. J. Favus, Ed., Raven Press, New York.

### Biological Testing

### General Methodology

### Drug Administration

Oral administration was by gavage in a vehicle comprising 1 mL/kg of body weight of a 20% aqueous solution of hydroxypropyl-β-cyclodextrin. Compound 1a was administered by gavage in a vehicle of 1 mL/kg of body weight of a 20% aqueous solution of cyclodextrin. The estrogen-treated control animals were administered 0.1 mg/kg/day 17α-ethynyl estradiol by gavage.

Subcutaneous administration of PTH(1-34) was by injection of an acidified saline vehicle (0.001 N HCl and 2% heated-inactivated rat serum in physiological saline (Butler Co., Columbus, Ohio, USA).

### Tissue Collection

Following treatment with the test compounds, the rats were anesthetized and subjected to cardiac puncture and euthanized by CO₂ inhalation. Uteri were removed and wet weight were determined on a Mettler balance to evaluate ovariectomy and efficacy of treatment with estrogen. Blood samples were allowed to clot at 4° C. for 2 hr before centrifugation at 2,000 g for 10 min. Serum were collected and stored at −70° C. before analysis. Serum cholesterol was assayed using a high performance calorimetric assay (Boehringer Mannheim Biochemicals, Indianapolis, Ind.). Tibia and femora were removed, cleaned of soft-tissue, fixed in 50% ethanol/ saline, and stored at 4° C. Vertebra L1-6 were removed and analyzed by QCT, histomorphometry, and biomechanics.

### X-Ray Bone Densitometry of Excised Rat Bones

The metaphysis of proximal tibiae were scanned longitudinally from baseline, using a 960A pQCT loaded with Dichte software version 5.2 (Norland/Stratec, Ft. Atkinson, Wis.), using the technique described by Sato et al., —1995, JPET 272:1252-1259; Sato 1995, Bone 17:157S-162S). Volumetric bone mineral density (BMD, mg/cm3), cross-sectional area (X-Area), voxel number, and mineral content (BMC, mg) were quantitated for the whole cross-section of the metaphysis. Sites of excised bones were analyzed at higher resolution, using a micro-CT (Stratec). Specifically, distal femora (below the condyles, see Sato et al. 1995) and L-3 vertebra (mid-cross section, Helterbrand et al., 1997, Bone 21:401-409) were analyzed using voxel dimensions of 50 ×50×1000 μm and 70×70×1000 μm, respectively.

### Histomorphometry

For histomorphometry, L-1 vertebra were trimmed, using a low-speed diamond saw (Buehler Ltd., Lake Bluff, Ill.) and fixed in 70% ethanol. Specimens were stained for 4 days in Villanueva osteochrome bone stain (Polysciences Inc., Warrington, Pa.), destained, dehydrated in a graded series of alcohols, and defatted in acetone. L-1 vertebra were then infiltrated with methyl methacrylate (as described by Schenk et al. 1984) and embedded in a 75 ml: 19 ml: 2.5 g mixture of methyl methacrylate: dibutyl phthalate: benzoyl peroxide (Kodak, Rochester, N.Y.) and polymerized at room temperature. Longitudinal sections (4 and 8 μm) were cut on a Reichert-Jung 2065 microtome (Magee Scientific Inc., Dexter, Mich.). The 4 μm sections were stained with 6% silver nitrate (Von Kossa stain) before coverslipping; the 8 μm thick sections were mounted unstained for dynamic measurements. Sections were glued onto slides dipped in 0.5% gelatin, dried overnight, and coverslipped with Eukitt.

Histomorphometric measurements were made using an Optiphot-2 fluorescence microscope (Nikon, Melville, N.Y.) and a semi-automatic digitizing system (SummaSketch III, Summagraphics Co., Seymour, Conn.; KSS Image Analysis, KSS Scientific Consultants, Magna Utah) coupled to a PowerPC 7100/66 (Apple Computer, Cupertino, Calif.), using the image capture functions of NIH Image 1.59 (NIH, Bethesda, Md.). For L-1, the entire marrow region within the cortical shell was measured to derive trabecular bone parameters. Specifically, measurements were made of cancellous bone volume (BV/TV, %), trabecular thickness (Tb.Th, μm), number (Tb.N, #/mm) and separation (Tb.Sp, μm), mineralizing surface, mineral apposition rate, and bone formation rate, as previously described (Frost 1983; Jee et al. 1985; Parfitt et al., 1987).

### Biomechanical Analyses

Bone strength was measured for the femoral neck, midshaft, and the L-6 vertebra. Femora were thawed before testing, and bone strength was measured on intact femora using a three point bending test. Load was applied midway between two supports that were 15 mm apart. The femora were positioned so the loading point was 7.5 mm proximal from the distal popliteal space and bending occurred about the medial-lateral axis. Specimens were tested in a saline bath at 37° C. Each specimen was submerged in the saline bath for three minutes before testing to allow equilibration of temperature. Load-displacement curves were recorded at a crosshead speed of 1 mm/sec using a servo-hydraulic materials testing machine (MTS Corp., Minneapolis, Minn.) and an x-y recorder (Hewlett Packard 7090A, Palo Alto, Calif.). The measurements included: cross-sectional moment of inertia (I), cortical thickness (t), ultimate load (Fᵤ), ultimate stress (σᵤ), Young's modulus (E), and toughness (u). These values were calculated as described previously (Turner and Burr 1993; Turner et al. 1996).

Femoral neck strength was measured by mounting the proximal half of the femur vertically in a chuck and applying downward force at a rate of 1 mm/sec on the femoral head until the neck failed. The ultimate load was calculated as the maximum force sustained by the femoral neck. All tests were done at room temperature using the MTS system.

Bone strength of L-6 vertebrae was measured after the posterior processes were removed and the ends of the centrum made parallel using a diamond wafering saw (Buehler Isomet, Evanston, Ill.). Ultimate stress (σᵤ) Young's modulus (E), and toughness (u) for each vertebra was measured in compression at a load rate of 50 N/sec using the MTS machine. The compressive load was applied through a pivoting platen to correct for nonparallel alignment of the faces of the vertebral body (Turner and Burr, 1993).

Specimens were tested in saline solution at 37° C. Ultimate stress was calculated as the maximum load divided by the gross cross-sectional area πAB/4, where A and B are the vertebral widths in the anterior-posterior and medial lateral directions. Young's modulus was calculated by multiplying stiffness times 4T/πAB, where T is the specimen thickness. Toughness was calculated as the area under the load-displacement curve divided by πABT/4.

### Statistical Methods

Data are presented as mean ±standard error of the mean. Precision was calculated by averaging the coefficient of variation (variability) as defined by standard deviation/mean for the specified rats. Group differences were assessed by analysis of variance (ANOVA) with pair-wise contrasts examined using primarily Fisher's protected least significant difference (PLSD) where the significance level for the overall ANOVA was p<0.05.

### Studies

In the first study, six-month old virgin Sprague-Dawley female rats weighing about 300 g each were maintained on a 12 hr light/12 hr dark cycle at 22° C. with ad lib access to food (TD 89222 diet containing 0.5% calcium and 0.4% phosphorus, Teklad, Madison, Wis., USA), Except for sham-operated control animals, bilateral ovariectomies were performed on the test animals. In the case of the sham-operated animals, the surgical ovariectomization technique was followed, but the ovaries were left intact to provide a control for assessing any effect which might be due to the surgical trauma.

The ovariectomized (OVX) animals were randomized and permitted to lose bone for one month before beginning treatment for the following three months.

The animal groups included: sham-operated animals(SHAM, n=8); bilaterally ovariectomized animals (OVX, n=7); bilaterally ovariectomized animals treated with 17α-ethynyl estradiol (Sigma Fine Chemicals, St. Louis, Mo., USA) dosed orally at a level of 0.1 mg/kg/day (n=7); and bilaterally ovariectomized animals treated with compound 1a at levels of 0.003, 0.03. 0.3 or 3 mg/kg/day, respectively, dosed orally (n=8 in each group).

SHAM and OVX control animals were orally dosed by gavage with the vehicle or 1 mL/kg of body weight of 20% hydroxypropyl-β-cyclodextrin (Aldrich Chemical Co., Milwaukee, Wis., USA). Estrogen-treated control animals were administered 0.1 mg/kg/day of 17α-ethynyl estradiol by gavage. Animals treated with various doses of 1a were administered the appropriate dose by gavage in 1 mL/kg of body weight of 20% cyclodextrin.

In a second study, virgin Sprague-Dawley rats were bilaterally ovariectomized (except for the sham-operated group), the ovariectomized animals randomized, and allowed to lose bone for one month prior to treatment in the manner described above. The animal groups, each containing 7-8 animals, included sham-operated animals (SHAM); bilaterally ovariectomized animals (OVX); bilaterally ovariectomized animals treated with 0.01, 0.3 or 1 mg/kg/day of compound 1a, respectively, for three months; bilaterally ovariectomized animals treated subcutaneously with PTH(1-34) at a dose of 10 μg/kg/day for three months; bilaterally ovariectomized animals treated subcutaneously with PTH(1-34) at a dose of 10 μg/kg/day and orally with compound 1a at a dose of 0.3 mg/kg/day, both compounds administered for three months; bilaterally ovariectomized animals treated subcutaneously with PTH(1-34) at a dose of 10 μg/kg/day for forty-five days followed by gavage administration of vehicle for forty-five days; bilaterally ovariectomized animals treated subcutaneously with PTH(1-34) at a dose of 10 μg/kg/day for forty-five days followed by oral administration of only compound 1a at a dose of 0.3 mg/kg/day for forty-five days; and bilaterally ovariectomized animals treated with 17α-ethynyl estradiol administered orally at a dose of 0.1 mg/kg/day.

### Results

### Effects of Compound 1a on body Weight and Uterine Weight

In the first study, ovariectomy was confirmed to increase body weight to significantly above Sham. Treatment with compound 1a at 0.03-3 mg/kg had no effect on body weight compared to OVX, and was significantly greater than Sham. By contrast, EE2 lowered body weight below OVX to Sham levels. Treatment with 10 μg/kg PTH (1-34) either alone or in combination with compound 1a also had little effect compared to OVX, and was greater than Sham. Body weight for the sequential combination of PTH (1-34) followed by compound 1a were significantly less than OVX but greater than Sham.

Ovariectomy was confirmed to decrease uterine wet weight compared to Sham. Treatment of animals with compound 1a at 0.003-3 mg/kg had no effect on uterine weight, while 0.1 mg/kg 17α-ethynyl estradiol increased uterine weight above OVX to Sham levels.

### Longitudinal Analysis of the Effects Compound 1a in Osteopenic Ovariectomized Rats

In the second study, the proximal tibial metaphysis was scanned longitudinally by pQCT for rats from baseline. As shown in FIG. 1, ovariectomy significantly reduced volumetric BMD by 20 and 25% compared to Sham (p<0.0001, Fisher's PLSD), respectively, by 1 month post-surgery (cf. FIG. 1).

Treatment, as indicated, was initiated after 1 month postovariectomy and continued for the following 3 months. Specifically, administration of compound 1a alone(closed triangles), PTH (1-34) alone (open triangles), in combination (closed squares), or in sequence (closed diamonds) were compared to OVX (open circles), Sham (closed circles), and estrogen (EE2, open diamonds) controls.

All three doses of compound 1a (0.01, 0.3, 1.0 mg/kg) prevented further bone loss and had BMD significantly greater than OVX at termination like 17α-ethynyl estradiol (0.1 mg/kg, EE2). These data show that 1a is able to prevent further reduction of bone induced by ovariectomy, like estrogen.

Rats adminstered PTH (1-34) for 45 days regained BMD to Sham levels (FIG. 1, open squares). However, when PTH (1-34) was discontinued, BMD decreased to significantly below Sham. Sequential studies in which rats were dosed with PTH (1-34) for 45 days before switching to compound 1a for the remainder of the study, showed that compound 1a prevented loss of BMD after discontinuation of PTH (1-34). That is, BMD were not different from Sham levels at termination (FIG. 1, closed diamonds).

In other rats treated continuously for 90 days with PTH (1-34) and compound 1a (10 μg/kg) BMD linearly increased to significantly beyond OVX, Sham, and Baseline levels, FIG. 1, closed squares). The combination of compound 1a (0.3 mg/kg) and PTH (1-34) increased BMD to levels significantly higher than any other group (p<0.00017 Fisher's PLSD). This combination increased BMD significantly faster and to higher levels than any agent alone, including PTH (1-34).

### Micro-CT Analyses of Femora and Vertebrae

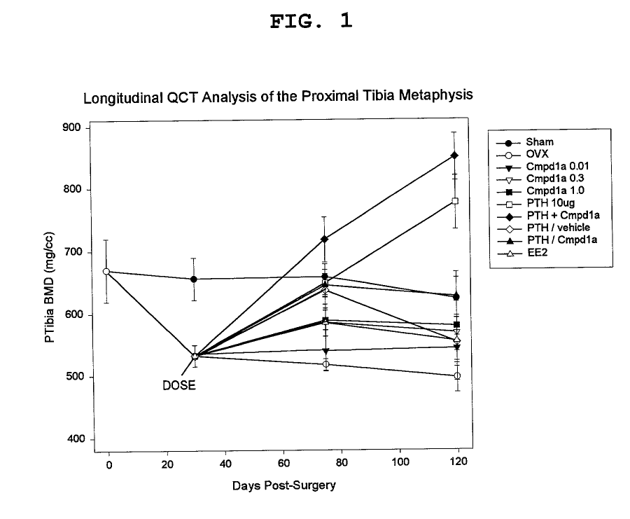

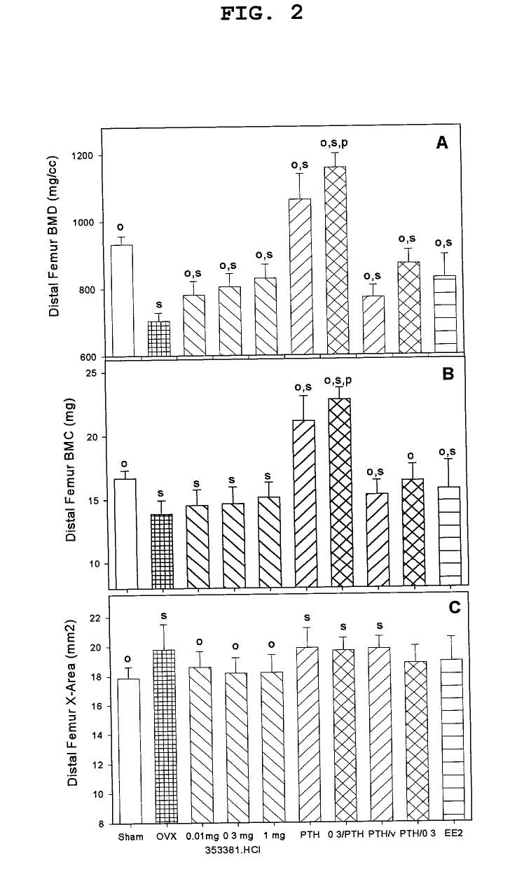

LY353381.HCl effects on the distal femur metaphysis in osteopenic rats was evaluated at 50×50 μm pixel resolution by micro-CT. Micro-CT largely confirmed observations made longitudinally in vivo. Specifically, compound 1a at 0.01-1 mg/kg prevented further bone loss in a manner similar to 0.1 mg/kg 17α-ethynyl estradiol. In sequential studies in which rats were dosed with PTH (1-34) for 45 days before switching to compound 1a, PTH/0.3 prevented the subsequent loss of BMD after discontinuation of PTH (1-34) (PTH/0). In other rats treated continuously with PTH (1-34), increased BMD was observed to levels significantly beyond OVX, Sham, and baseline levels. The combination of compound 1a (0.3 mg/kg) and PTH (1 -34) increased BMD to levels significantly higher than any other group, including PTH (1-34) (p<0.0001, Fisher's PLSD (FIG. 2).

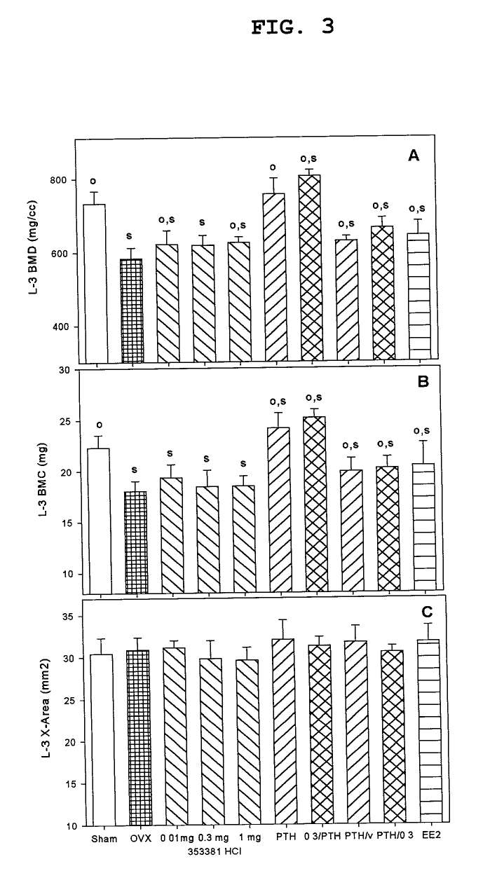

Micro-CT analysis of L-3 vertebra at 70×70 μm pixel resolution showed marginal effects of compound 1a at 0.01-1 mg/kg and 0.1 mg/kg 17α-ethynyl estradiol on spinal BMD or BMC, compared to OVX. However, continuous dosing with PTH (1-34) improved BMD to significantly above OVX, while the combination of compound 1a (0.3 mg/kg) and PTH (1-34) increased BMD and BMC to significantly above both OVX and Sham. These data show that the compound 1a/PTH (1-34) combination increases bone beyond either treatment alone in the appendicular and axial skeleton (FIG. 3).

### Static and Dynamic Histomorphometry of LY353381.HCI Effects

For the second study, higher resolution analyses of the effects of compound 1a on L-1 vertebra were obtained by histomorphometry. ovariectomy decreased trabecular bone volume (BV/TV), trabecular thickness, and trabecular number compared to Sham. Compound 1a and EE2 had little effect on BV/TV, trabecular thickness, or trabecular number which were not different from OVX. However, PTH (1-34) improved BV/TV and trabecular thickness above OVX, while the combination of compound 1a (0.3 mg/kg) and PTH (1-34) increased BV/TV significantly beyond Sham and PTH (1-34) alone.

Discontinuation of PTH (1-34) decreased BV/TV and trabecular thickness to below Sham, but switching to compound 1a at 45 days prevented this loss.

TABLE 1
Static and Dynamic Histomorphometry of L-1

| | BV/TV | Tb.Th | Tb.N | Tb.Sp | MS/BS | MAR | BFR/BS | BFR/TV |
|---|---|---|---|---|---|---|---|---|
| Group | (%) | (μ) | (#/mm) | (μ) | (%) | (μ/d) | (μ/d × 100) | (%/y) |
| Sham | 35.1 ± 5.3^{o} | 49.2 ± 7.5^{o} | 7.19 ± 79^{o} | 91.5 ± 14.9^{o} | 12.37 ± 1.51^{o} | 0.70 ± .06 | 8.63 ± .95^{o} | 37.72 ± 5.11 |
| OVX | 21.1 ± 6.3^{s} | 40.8 ± 8.2^{s} | 5.23 ± 1.5^{s} | 168.9 ± 76^{s} | 19.19 ± 2.78^{s} | 0.72 ± .12 | 13.95 ± 3.72^{s} | 44.78 ± 18.6 |
| Cmpd 1a | | | | | | | | |
| 0.01 mg/kg | 24.4 ± 5.9^{s} | 42.4 ± 6 | 5.77 ± 1.2^{s} | 137.6 ± 37^{s} | 15.90 ± 2.21^{s.o} | 0.72 ± .06 | 11.45 ± 1.9 | 40.30 ± 11.41 |
| 0.3 mg/kg | 22.3 ± 3^{s} | 36.8 ± 3^{s} | 6.07 ± .87^{s} | 131 ± 23^{s.o} | 14.47 ± 1.78^{o} | 0.66 ± .11 | 9.48 ± 1.43^{o} | 35.51 ± 9.14 |
| 1 mg/kg | 22.1 ± 5.2^{s} | 42.4 ± 7.3 | 5.24 ± .82^{s} | 153.1 ± 35^{s} | 13.05 ± 2.44^{s} | 0.72 ± .08 | 9.44 ± 2.4^{o} | 29.94 ± 8.7 |
| PTH | 38.9 ± 9^{o} | 63.8 ± 11^{s,o} | 6.07 ± .86^{s} | 104.1 ± 29^{o} | 24.82 ± 2.62^{s,o} | 1.0 ± .11^{s,o} | 25.1 ± 5^{s,o} | 94.5 ± 29.7^{s.o} |
| PTH + 1a | 48.5 ± 6.8^{s,o} | 71.2 ± 9.3^{s,o} | 6.84 ± .73^{o} | 76.4 ± 15^{o} | 29.91 ± 6.17^{s,o} | 1.0 ± .12^{s,o} | 30 ± 7.7^{s,o} | 123.6 ± 28.7^{s,o} |
| PTH/vehicle | 20.1 ± 3.9^{s} | 39.4 ± 7^{s} | 5.16 ± .98^{s} | 160 ± 32^{s} | 19.62 ± 4.88^{s} | 0.89 ± .09^{s,o} | 17.68 ± 5.9^{s} | 55.17 ± 20.3^{s} |
| PTH/1a | 27.8 ± 4.7^{s.o} | 46.9 ± 5.5 | 5.90 ± .6^{s} | 124.1 ± 19.8^{o} | 16.89 ± 2.33^{s} | 0.74 ± .12 | 12.66 ± 3.1^{s} | 45.43 ± 11.8 |
| EE2 | 21.8 ± 4.1^{s} | 35.3 ± 5.6^{s} | 6.20 ± .66 | 127.5 ± 17.5 | 14.97 ± 3.07^{o} | 0.58 ± .05^{s.o} | 8.76 ± 1.99^{o} | 32.76 ± 6.9 |
| ^{a}As indicated, treatment groups from experiment 1 included Sham, OVX, Compound 1a alone (0.01, 0.3, 1.0 mg/kg), PTH (1-34) alone (PTH, 10 μg/kg), PTH (1-34) in combination with Compound 1a (PTH + 1a), PTH (1-34) for 45 days followed by vehicle (PTH/vehicle), PTH (1-34) for 45 days followed by Compound 1a (PTH/1a), or 17α ethynyl estradiol (EE2, 0.1 mg/kg). Data are mean followed by SEM (n = 7-8). | | | | | | | | |
| #Significant differences from Sham or OVX are depicted as “s”, “o”, respectively (p < 0.05, Fisher's PLSD). | | | | | | | | |

### Examination of Dynamic Parameters Confirmed the Ovariectomy Stimulation of Mineralized Surface (MS/BS) and Bone Formation Rate (BFR/BS).

Compound 1a decreased MS/BS and BFR in a dose-dependent manner to Sham and EE2 levels. However, MAR for compound 1a 0.01-1 mg/kg were not different from OVX, while EE2 lowered MAR to below OVX, Sham, and compound 1a (0.01-1 mg/kg). PTH (1-34) increased MS/BS, MAR, BFR/BS and BFR/TV to significantly above Sham and OVX. PTH (1 -34) in combination with compound 1a increased MS/BS, BFR/BS and BFR/TV to above PTH (1-34) alone. Discontinuation of PTH (1-34) at 45 days (PTH/v) lowered MS/BS, BFR/BS and BFR/TV to OVX levels. Switching to compound 1a at 45 days (PTH/0.3) lowered MAR, BFR/BS and BFR/TV to below that of the PTH/v group.

These data show that compound 1a does decrease bone turnover like EE2, but suppresses bone formation (MAR) to a lesser extent than EE2. Additionally, the group to whom PTH (1-34) and compound 1a were sequentially administered had higher bone formation and mineralization activity than either treatment, alone.

### Biomechanical Analysis of Bone Quality

The femora diaphysis was evaluated by 3 point-bending analysis of the mid-shaft. Compound 1a improved load to failure (Fᵤ) and toughness in a dose-dependent manner to above OVX and not different from Sham at 1 mg/kg, as did EE2 at 0.1 mg/kg. PTH (1-34) treatment for 45 and 90 days improved Fᵤ to above OVX, but only the compound 1a/PTH (1-34) combination increased Fᵤ to significantly above Sham. No difference in the Young's modulus were observed between groups.

Measurement of the cortical thickness for compound 1a and EE2 treated animals showed that thickness for both were intermediate between Sham and OVX. Interestingly, PTH treatment for 90 days increased cortical thickness above OVX but only the compound 1a/PTH (1-34) combination increased cortical thickness above Sham. Discontinuation of PTH (1-34) after 45 days lowered cortical thickness to below Sham, but switching treatment to compound 1a prevented this decrease.

TABLE 2
Biomechanical Analyses of the Femora Mid-shaft

| Group | t | I | Fᵤ | σᵤ | E | u |
|---|---|---|---|---|---|---|
| Sham | .715 ± .012 | 4.51 ± .23 | 187 ± 5^{o} | 230 ± 5^{o} | 9645 ± 522 | 4.76 ± .42^{o} |
| OVX | .607 ± .017^{s} | 4.75 ± .22 | 144 ± 5^{s} | 173 ± 7^{s} | 8289 ± 608 | 2.76 ± .26^{s} |
| Cmpd 1a | | | | | | |
| 0.01 | .635 ± .020^{s,o} | 4.35 ± .21 | 160 ± 5^{s} | 199 ± 5^{s,o} | 9081 ± 446 | 3.72 ± .26^{s,o} |
| 0.3 | .654 ± .012^{s,o} | 4.05 ± .17^{o} | 164 ± 4^{s,o} | 217 ± 5^{o} | 8844 ± 431 | 4.13 ± .24^{o} |
| 1 | .671 ± .010^{s,o} | 4.17 ± .21 | 174 ± 4^{o} | 228 ± 9^{o} | 10087 ± 520 | 4.52 ± .25^{o} |
| PTH. | .739 ± .023^{o} | 4.86 ± .26 | 202 ± 7^{o} | 234 ± 9^{o} | 9854 ± 597 | 5.24 ± .52^{o} |
| 0.3 + PTH | .775 ± .014^{s,o} | 5.01 ± .17 | 215 ± 6^{s,o} | 237 ± 7^{o} | 9839 ± 626 | 5.29 ± .39^{o} |
| PTH/v | .630 ± .010^{s} | 4.72 ± .17 | 171 ± 4^{o} | 205 ± 6^{s,o} | 8516 ± 580 | 3.81 ± .24^{s,o} |
| PTH/ | .705 ± .130^{o} | 4.21 ± .20 | 181 ± 4^{o} | 232 ± 10^{o} | 10350 ± 777 | 4.48 ± .23^{o} |
| Cmpd 1a | | | | | | |
| 0.3 | | | | | | |
| EE2 | .686 + .015^{o} | 4.42 ± .33 | 182 ± 11^{o} | 225 ± 13^{o} | 10002 ± 844 | 4.62 ± .52^{o} |

a Cortical bone properties of the femora diaphysis were examined by 3-point-bending to measure the cortical thickness (t), moment of inertia (I), ultimate force (Fᵤ), strength (σᵤ, Young's modulus (E), and toughness (u). Data are mean followed by SEM (n = 7-8). Significant differences from Sham or OVX are depicted as “s”, “o”, respectively (p < 0.05, Fisher's PLSD).

Quality of the proximal femur and L-6 vertebra were evaluated by femora neck shear and compression testing, respectively. Ultimate load for the femora neck showed no differences between groups, except for PTH (1-34) alone and in combination with compound 1a which were both higher than OVX and Sham. OVX vertebra were significantly weaker than Sham (Su). Compound 1a improved vertebral strength (Su) and toughness in a dose-dependent manner to above OVX and were not different from EE2 or Sham. PTH treatment for 90 days, either alone or in combination with compound 1a, increased Su and toughness to above OVX and Sham levels. Discontinuation of PTH (1-34) after 45 days lowered Su to below Sham, but switching treatment to compound 1a prevented this decrease.

TABLE 3
Biomechanical Analyses of the Proximal Femur and Lumbar Vertebra

| Group | Neck Fᵤ | Vert σᵤ | Vert E | Vert u |
|---|---|---|---|---|
| Sham | 119 ± 5.5 | 33.3 ± 3.8^{o} | 660 ± 122 | 1.91 ± 0.44 |
| OVX | 112 ± 5.6 | 21.0 ± 2.2^{s} | 578 ± 66 | 0.74 ± 0.15 |
| Cmpd. 1a | | | | |
| 0.01 | 112 ± 4.7 | 24.8 ± 2.3^{s} | 540 ± 72 | 1.72 ± 0.44 |
| 0.3 | 114 ± 5.1 | 28.3 ± 1.9^{o} | 436 ± 76 | 1.97 ± 0.37 |
| 1 | 117 ± 3.1 | 27.8 ± 1.5^{o} | 384 ± 30 | 2.30 ± 0.29^{o} |
| PTH | 148 ± 2.6^{s,o} | 45.5 ± 2.4^{s,o} | 686 ± 77 | 3.68 ± 1.10^{s,o} |
| 0.3 + PTH | 146 ± 6.4^{s,o} | 50.6 ± 2.0^{s,o} | 693 ± 102 | 4.50 ± 0.79^{s,o} |
| PTH/v | 116 ± 6.2 | 24.2 ± 2.5^{s} | 524 ± 109 | 1.16 ± 0.22 |
| PTH/1a | 118 ± 4.4 | 29.4 ± 1.9^{o} | 450 ± 47 | 1.79 ± 0.27 |
| 0.3 | | | | |
| EE2 | 110 ± 6.1 | 28.1 ± 2.6^{o} | 579 ± 94 | 1.44 ± 0.37 |

a Proximal femora were loaded to failure in shear to measure the ultimate force (Neck Fᵤ). L-5,6 vertebra were loaded to failure in compression to measure vertebral strength (Vert Sᵤ), Young's modulus (Vert E), and toughness (Vert u). Data are mean followed by SEM (n = 8). Significant differences from Sham or OVX are depicted as “s”, “o”, respectively (p < 0.05, Fisher's PLSD).

These data show that compound 1a prevents the loss of bone, and also demonstrate an advantage of the combination and sequential use of compound 1a with PTH (1-34) on both cortical and trabecular bone sites.

I claim:

1. A method of building bone in a patient in need of such treatment comprising co-administering to a patient in need of such treatment a therapeutically effective amount of parathyroid hormone and a compound or pharmaceutically acceptable salt thereof of the formula 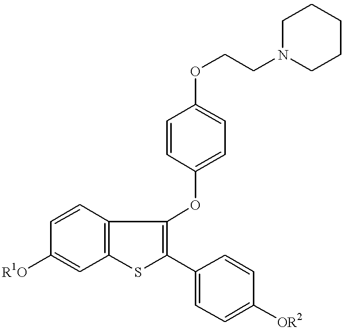
wherein R¹ and R² are independently selected from the group consisting of hydrogen and alkyl of one to six carbon atoms.

2. A method as defined by claim 1 wherein R¹ and R² are independently selected from hydrogen and methyl.

3. A method as defined by claim 1 wherein R¹ and R² are both hydrogen.

4. A method as defined by claim 2 wherein R¹ is hydrogen and R² is methyl.

5. A method as defined by claim 1 wherein said parathyroid hormone is human parathyroid hormone (1-84) (Sequence ID No. 1).

6. A method as defined by claim 1 wherein said parathyroid hormone is human parathyroid hormone (hPTH 1-31) (Sequence ID No. 2).

7. A method as defined by claim 1 wherein said parathyroid hormone is human parathyroid hormone (hPTH 1-34) (Sequence ID No. 3).

8. A method as defined by claim 1 wherein said parathyroid hormone is human parathyroid hormone (hPTH 1-38) (Sequence ID No. 4).

9. A method as defined by claim 1 wherein said parathyroid hormone is PTH related hormone (PTHrP 1-34) (Sequence ID No. 5).

10. A method as defined by claim 1 wherein said parathyroid hormone is [Glu²² Leu²³, Glu²⁵, Lys²⁶, Leu²⁸, Glu²⁹, Lys³⁰ Leu³¹, homo-Ser³⁴ (lactam)]PTHrP (Sequence ID No. 6).

11. A method as defined by claim 1 wherein said parathyroid hormone is [Ala²¹, Glu²², Leu²³, Glu²⁵ Lys²⁶, Leu²⁸, Glu²⁹, Lys³⁰, Leu³¹]PTHrP (Sequence ID No. 7).

12. A method as defined by claim 1 wherein said parathyroid hormone is [Glu²², Leu²³, Glu²⁵, Lys²⁶, Leu²⁸, Glu²⁹, Lys³⁰, His³¹, Thr³², Ala³⁴, des-Ala³⁴]PTHrP (Sequence ID No. 8).

13. A method as defined by claim 1 wherein said parathyroid hormone is [Leu²⁷ cyclo(Glu²²-Lys²⁶)]hPTH(1-34) (Sequence ID No. 9).

14. A method as defined by claim 1 wherein said parathyroid hormone is Leu²⁷ cyclo(Lys²⁶-Asp³⁰)]hPTH(1-31) (Sequence ID No. 10).

15. A method as defined by claim 1 wherein said parathyroid hormone is [Leu⁸, Asp¹⁰, Lys¹¹, Ala¹⁶, Gln¹⁸, Thr³³, Ala³⁴]hPTH(1-34) (Sequence ID No. 11).

16. A method as defined by claim 1 wherein said co-administration is simultaneous.

17. A method as defined by claim 1 wherein said co-administration is concurrent.

18. A method as defined by claim 1 wherein said co-administration is sequential.

19. A method of building bone in a patient in need of such treatment comprising co-administering a therapeutically effective amount parathyroid hormone and a compound of formula 1a 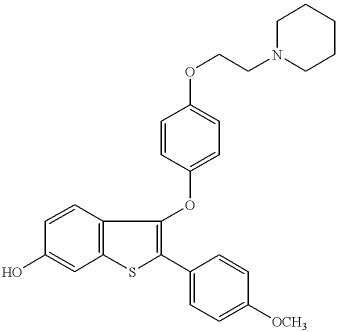
1a
or a pharmaceutically acceptable salt thereof, for a period sufficient to raise bone mass in the patient to within one standard deviation of normal, followed by cessation of the administration of parathyroid hormone.

20. The method as defined by claim 20 wherein said parathyroid hormone is human parathyroid hormone (hPTH 1-34) (Sequence ID No. 3).


SEQUENCE LISTING (12 sequences)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 2 - protein, 31 aa, Homo sapiens
SVSEIQLMHNLGKHLNSMERVEWLRKKLQDV

SEQ ID NO: 3 - protein, 34 aa, Homo sapiens
SVSEIQLMHNLGKHLNSMERVEWLRKKLQDVHNF

SEQ ID NO: 4 - protein, 38 aa, Homo sapiens
SVSEIQLMHNLGKHLNSMERVEWLRKKLQDVHNFVALG

SEQ ID NO: 5 - protein, 34 aa, Homo sapiens
AVSEHQLLHDKGKSIQDLRRRFFLHHLIAEIHTA

SEQ ID NO: 6 - protein, 34 aa, Homo sapiens
  Xaa at position 34 is homo SER (lactam).
AVSEHQLLHDKGKSIQDLRRRELLEKLLEKLHTX

SEQ ID NO: 7 - protein, 34 aa, Homo sapiens
AVSEHQLLHDKGKSIQDLRRAELLEKLLEKLHTA

SEQ ID NO: 8 - protein, 34 aa, Homo sapiens
AVSEHQLLHDKGKSIQDLRRRELLEKLLEKLHTA

SEQ ID NO: 9 - protein, 31 aa, Homo sapiens
AVSEIQFMHNLGKHLSSMERVEWLRKLLQDV

SEQ ID NO: 10 - protein, 31 aa, Homo sapiens
AVSEIQFMHNLGKHLSSNERVEWLRKLLQDV

SEQ ID NO: 11 - protein, 34 aa, Homo sapiens
AVSEIQFLHDKGKHLASQERVEWLRKKLQDVHTA

SEQ ID NO: 12 - protein, 40 aa, Homo sapiens
AVSEHQLLHDKGKSIQDLRRRFFLHHLIAEIHTAEIRATS